(19) United States
(12) Patent Application Publication
(10) Pub. No.: US 2024/0325485 A1
(43) Pub. Date: Oct. 3, 2024

(54) COMPOSITION COMBINATION CONTAINING ENRICHED HILL SHADDOCK OIL

(71) Applicant: JIANGNAN UNIVERSITY, Wuxi (CN)

(72) Inventors: Ruijie LIU, Wuxi (CN); Yiwen GUO, Wuxi (CN); Zhu ZHU, Wuxi (CN); Ming CHANG, Wuxi (CN)

(21) Appl. No.: 18/610,110

(22) Filed: Mar. 19, 2024

(30) Foreign Application Priority Data
Mar. 30, 2023 (CN) 202310325724.2

(51) Int. Cl. A61K 36/82; A61K 31/232

(52) U.S. Cl. CPC A61K 36/82; A61K 31/232

(57) ABSTRACT

A combination containing enriched hill shaddock oil (HSO) is provided to prevent or alleviate inflammatory bowel disease (IBD). The composition combination includes 30% to 50 wt % of branched chain fatty acid (BCFA) glycerides and 50% to 70 wt % of the oil-tea seed oil. The composition combination has significant anti-inflammatory effect on intestinal epithelial cells. Secretion of inflammatory factors is regulated to reduce the production of products of cellular inflammatory reactions. By adding HSO, intestinal tissue damage is alleviated, repair of the intestinal barrier is promoted, and the remission period of IBD is prolonged. The BCFA glycerides are formed by combining diacylglycerol with triacylglycerol and facilitate digestion, absorption, and metabolism, allowing relief of fat digestion and absorption disorders in patients suffering from IBD. In addition, the composition combination has enriched mono-unsaturated fatty acids, enabling the composition combination to be stable, facilitating regulation of human physiological functions and improving metabolism.

### CROSS REFERENCE TO RELATED APPLICATIONS

The present disclosure is a continuation application of 202310325724.2, filed on Mar. 30, 2023, and its contents are incorporated herein in their entirety.

### TECHNICAL FIELD

The present disclosure relates to the field of formulated food for special medical uses, and in particular to a combination containing enriched hill shaddock oil (HSO).

### BACKGROUND

An inflammatory bowel disease (IBD) is a chronic and non-specific intestinal inflammatory disease with a complex pathogenesis that cannot be easily cured. Due to its poor prognosis, pharmacological treatments may not achieve desired effects and can even cause adverse effects. Therefore, additional preventive or supplementary strategies may be necessary to treat IBD effectively. Appropriate dietary and nutritional interventions can help to improve malnutrition and the prognosis of patients with IBD, which is crucial for enhancing treatment outcomes.

Fat is one of the three major energy sources in humans and plays an important role in maintaining the health of humans. Rational fat intake and fat composition are important aspects of dietary and nutritional interventions. However, in the field of IBD, no further research has been done on the influence of fat components in medical formulas on inflammation. Therefore, developing a combination containing enriched HSO for preventing or alleviating IBD and providing enough nutrition to patients is an urgent technical problem to be solved.

### SUMMARY OF THE DISCLOSURE

The present disclosure aims to provide a combination enriched with HSO for preventing or alleviating IBD. The combination has significant anti-inflammatory effects on intestinal epithelial cells. The combination regulates the secretion of inflammatory factors to reduce the production of products of cellular inflammatory reactions. The HSO works in combination with other compositions to reduce damage to intestinal tissues, facilitate repair of intestinal barriers, and prolong the remission period of IBD.

In order to achieve the above objectives, the present disclosure provides a combination enriched with HSO, for preventing or relieving an IBD. The combination enriched with HSO includes 30% to 50% by weight of branched chain fatty acid (BCFA) glycerides and 50% to 70% by weight of HSO.

In some embodiments, the BCFA glycerides include a diacylglycerol (DAG) and a triacylglycerol (TAG), and the mass ratio of the DAG to the TAG is in the range of 0:3 to 2:1. In some embodiments, the mass ratio of DAG to TAG is 1:1.

In some embodiments, the BCFA glycerides are fatty acids with 8 to 20 carbon atoms.

In some embodiments, the fatty acids comprise anteiso-BCFAs and iso-BCFAs; the mass ratio of the anteiso-BCFAs to the iso-BCFAs is in the range of 2:1 to 4:1.

In some embodiments, in mass percentages, the fatty acids comprise: 80% to 90% of oleic acid, 2% to 9% of linoleic acid, 0.05% to 1.5% of linolenic acid, 3% to 8% of palmitic acid, and 1% to 5% of stearic acid.

In some embodiments, in mass percentages, the fatty acids comprise: 82% to 87% of oleic acid, 3% to 7% of linoleic acid, 0.1% to 1% of linolenic acid, 4% to 7% of palmitic acid, and 1% to 3% of stearic acid.

In some embodiments, in mass percentages, the volatile flavor compositions of the HSO comprise the following in mass percentage: 5% to 8% of 2-methylpyrazine; 6% to 10% of 2-ethyl-6-methylpyrazine; 9% to 14% of furaldehyde; 14% to 18% of 5-methyl furfural; 2% to 5% of 4, 6-sulfadimidine; 0.1% to 2% of 2-acetyl pyrrole; 10% to 20% of alkanes; 10% to 20% of aldehydes; 10% to 18% of acids; 2% to 5% of alcohols; 0.5% to 3% of ketones; 1% to 5% of phenols; and 1% to 5% of esters.

In another aspect, the present disclosure provides a method for preparing a combination containing with enriched HSO in any of the above embodiments. The method includes:
Mixing the TAG and the DAG according to the mass ratio to obtain a first mixture, heating the mixture, stirring the mixture, and obtaining the BCFA glycerides; and then stopping the heating, adding the HSO to the BCFA glycerides to obtain a second mixture, stirring and cooling it to room temperature to obtain the combination containing enriched HSO.

In some embodiments, during the process of mixing the TAG and DAG according to the mass ratio to obtain a first mixture, the mixture is heated and stirred to produce BCFA glycerides. The heating rate is 3° C./min, and the temperature of the first mixture is increased to reach 30° C. to 40° C. The stirring is performed at 100 to 120 rpm for 15 to 20 minutes to ensure the mixture is uniform. Once the stirring is complete, the BCFA glycerides are obtained.

In some embodiments, when stopping the heating, HSO is added to the BCFA glycerides to obtain a second mixture. The second mixture is stirred at a speed of 100 rpm to 120 rpm for 15 minutes, after which the stirring is stopped. The second mixture is then cooled to room temperature at a rate of 3° C. per minute, resulting in the composition combination containing enriched HSO. The heating is stopped during this process. The HSO is added to the BCFA glycerides during the second stirring, which is then stopped after 15 minutes to obtain the second mixture. The second mixture is cooled down to room temperature at a rate of 3° C. per minute, resulting in the combination containing enriched HSO.

In some embodiments, the combination containing enriched HSO can be made into drops, solid powder, capsules, and so on.

In yet another aspect, the present disclosure provides a method of preventing or alleviating IBD. In the method, the combination containing enriched hill shaddock oil (HSO), as described in the above aspect, is applied as fat to a patient with the inflammatory bowel disease (IBD); or incorporating the combination containing enriched HSO, as described in the above aspect, into food provided for a patient with the IBD.

The following technical effects are achieved.

The BCFA in the present disclosure has a special structure and has a significant anti-inflammatory effect on human intestinal epithelial cells. The production of cellular inflammatory reactions is reduced by regulating the secretion of inflammatory factors. The BCFA and HSO work cooperatively to reduce damage to intestinal tissues, improve repair of intestinal barriers, and prolong remission period of IBD.

The main volatile flavor components of HSO in the present disclosure are heterocyclic compounds such as pyrazines. However, the main flavor components of tea oils from other regions are aldehydes and acids. The special rich aroma carried by HSO enables it to eliminate the unpleasant odor generated by some BCFA.

The compositions of the present disclosure are reasonable and can be easily digested and absorbed, which induces relief from IBD. These fatty acid compositions may be used as fat or as a fat component in formulated food specifically for patients suffering from IBD.

### BRIEF DESCRIPTION OF THE DRAWINGS

In order to more clearly illustrate the technical solutions of the embodiments of the present disclosure, the accompanying drawings used to describe the embodiments will be briefly introduced below. It is apparent that the following accompanying drawings are only some of the embodiments of the present disclosure, and any ordinary skilled person in the art may obtain other accompanying drawings according to the following drawings without making any creative work.

FIG. 1 shows the sensory evaluation of a combination containing enriched HSO, as per an embodiment of the present disclosure.

FIG. 2 is a column chart showing myeloperoxidase (MPO) activities in colon tissues of mice in a blank control group, in an induction modeling group, in the embodiment I group, in the embodiment II group, in the embodiment III group, in the embodiment IV group, and in the embodiment V group.

FIG. 3 (a) shows the detection results of the inflammation-related gene IL-18 in RAW264.7 cells, as per an embodiment of the present disclosure.

FIG. 3 (b) shows the ELISA results of the inflammation-related gene IL-17 in RAW264.7 cells, as per an embodiment of the present disclosure.

### DETAILED DESCRIPTION

In order to make the objectives, features and advantages of the present disclosure more obvious and understandable, specific implementations of the present disclosure will be described in detail in the following by referring to specific embodiments.

Many specific details are described in the following in order to facilitate a comprehensive understanding of the present disclosure. However, the present disclosure can be implemented in other ways different from the description herein. Any ordinary skilled person in the art may perform promotions without departing from the essence of the present disclosure. Therefore, the present disclosure is not limited by the embodiments disclosed hereinafter.

In the embodiments of the present disclosure, a BCFA refers to a class of fatty acids that have one or more branched chains (mainly methyl groups) on their carbon skeleton. This class of fatty acids includes both mono-branched-chain fatty acids and multi-branched-chain fatty acids and they are typically saturated fatty acids. When a methyl group of a branched chain is connected to the second-to-last carbon atom of the carbon skeleton, the BCFA is named an iso-BCFA. When a methyl group of a branched chain is connected to the third-to-last carbon atom of the carbon skeleton, the BCFA is named an anteiso-BCFA. BCFA has a special structure, and food usually has a lower amount of BCFA. BCFA has unique physiological functions. Intake of BCFA facilitates the establishment of intestinal bacterial flora, preventing necrotizing enterocolitis (NEC).

In the embodiments of the present disclosure, the HSO is a type of Camellia oleifera (C. oleifera) oil obtained by processing C. oleifera plants grown in Hainan Province. Due to historical and cultural factors, Hainan locals refer to the locally grown C. oleifera oil as HSO. It has a particularly aromatic odor and is rich in unsaturated fatty acids. Additionally, the unique climatic conditions in Hainan Province result in differences among the seeds, leading to a better smell, taste, and nutritional value of HSO compare to C. oleifera oil grown in mainland China. The aromatic odor of C. oleifera oil grown in the Hainan Province is distinctive, mainly due to the volatile flavor components of HSO, which are heterocyclic compounds, such as pyrazines. In contrast, the volatile flavor components of C. oleifera oil grown in other provinces of China are primarily aldehydes and acids.

In the embodiments of the present disclosure, IBD substantially includes Crohn's Disease (CD) and Ulcerative colitis (UC).

Specific detection methods performed in the embodiments of the present disclosure are described in the following.

1. Determination of fatty acids in the combination containing enriched HSO.

Analysis of the fatty acid composition is performed with reference to the “GB 5009.168-2016 National Standard for Food Safety-Determination of Fatty Acids in Food”.

2. Determination of the volatile flavor components in the combination containing enriched HSO.

The composition of the combination containing enriched HSO is used as a raw material. A solid-phase micro-extraction (SPME) device is used to perform headspace sampling, and the volatile flavor components are determined by performing gas chromatography-mass spectrometry (GC-MS).

3. Sensory evaluation of the flavor of the combination containing enriched HSO

To perform sensory evaluation of the flavor of the combination containing enriched HSO, a group of professionals is trained. From this group, 20 professionals are selected to evaluate various senses of each sample and give scores based on evaluation items. The evaluation rules are shown in Table 1.

TABLE 1
Sensory evaluation table for the combination containing
enriched HSO to prevent or relieve the IBD

| | Levels | | | | | | |
|---|---|---|---|---|---|---|---|
| | Very | | | | Very | | Total |
| Evaluation factors | strong | strong | moderate | weak | weak | none | score |
| Glossiness (glossiness of the | 6 | 5 | 4 | 3 | 2 | 1 | 21 |
| combination containing | | | | | | | |
| enriched HSO) | | | | | | | |
| Odor (intensity of the special | 10 | 8 | 6 | 4 | 2 | 1 | 31 |
| burnt smell of the | | | | | | | |
| combination containing | | | | | | | |
| enriched HSO) | | | | | | | |
| Taste (durability of the special | 8 | 6 | 5 | 4 | 3 | 1 | 27 |
| taste of the combination | | | | | | | |
| containing enriched HSO) | | | | | | | |
| Tissue morphology | 6 | 5 | 4 | 3 | 2 | 1 | 21 |
| (transparency of the | | | | | | | |
| combination containing | | | | | | | |
| enriched HSO) | | | | | | | |

4. Animal experiments

Using 50 five-week-old SPF-grade C57BL/6J mice, the mice were purchased and housed under standard conditions in the animal facility (temperature: 24-25° C., humidity: 70-75%, light cycle: 12 hours), with free access to food and water, and bedding changed 1-2 times per week. After a one-week adaptation period, the mice were randomly divided into a normal control group, an induction modeling group, and three intervention groups, each consisting of 10 mice.

After starting the experiment, the combination containing enriched HSO, which is made based on the formula of the present disclosure, is used to stomach-lavage each mouse in the three intervention groups once a day (0.5 ml/100 g). Each mouse in the remaining two groups is stomach-lavage with an equal volume of distilled water. The stomach lavage is performed for one week. Only the mice in the normal control group drink water at will, and the mice in the remaining groups are continued to be intervened in the same way as the intervention performed during the first week. At the same time, the mice are observed for any symptom of loose stools, diarrhea, and blood in stools, in order to determine whether the model is successfully established. When the model establishment is completed, the mice in the induction modeling group and the experimental groups are allowed to drink water at will. The mice in each group are fed normally and provided with an equal volume of the corresponding lavaged substances for one week. The colon of each mouse from each group is collected, and the MPO activity of the colon is determined by following the instructions of the kit.

5. Cellular experiments

Peritoneal macrophages (RAW264.7) that are growing well and are in a logarithmic phase are obtained. The number of cells is counted using a blood cell counting plate. 6×105 cells are inoculated in each well of a 6-well plate (3 mL/well). After 24 hours of incubation, gram-negative bacterial outer membrane lipopolysaccharide (LPS) is added to each well for stimulation to establish cell models. A normal control group and an inflammation model group are set up, each group with triplicates. The combination containing enriched HSO is embedded with whey isolate protein, resulting in a final concentration of 200 μg/mL of the combination containing enriched HSO in each well. After 2 hours of pretreatment, the cells are further treated with 1 μg/ml of LPS for 24 hours. Each group has triplicates. The effects on the secretion of cytokines IL-1B and IL-17 caused by the composition combination of each embodiment are determined using ELISA kits.

### Embodiment 1 (S1)

The combination containing enriched HSO for preventing or alleviating the IBD includes the following compositions, by weight percent:

40% of BCFA glycerides and 60% of HSO. The BCFA glycerides includes: DAG and TAG, in a mass ratio of 1:1. Additionally, a mass ratio of anteiso-BCFAs: iso-BCFAs is 3:1.

The TAG and DAG in the above mass ratio is added to a reactor, mixed, and stirred. The temperature of the reactor is increased at a rate of 3° C./min to reach 35° C. The stirring is performed at a speed of 100 rpm for 20 minutes. When the stirring is finished, a mixed BCFA glyceride is obtained. Furthermore, the temperature increase is stopped, and a second stirring is performed at a speed of 100 rpm. When the second stirring is finished, the oil-tea seed oil is added to the reactor, and a third stirring is performed for 15 minutes. Furthermore, the third stirring is stopped, and the temperature of the reactor is cooled down to room temperature at a rate of 3° C./min. The third stirring is stopped, and the combination containing enriched HSO is obtained.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

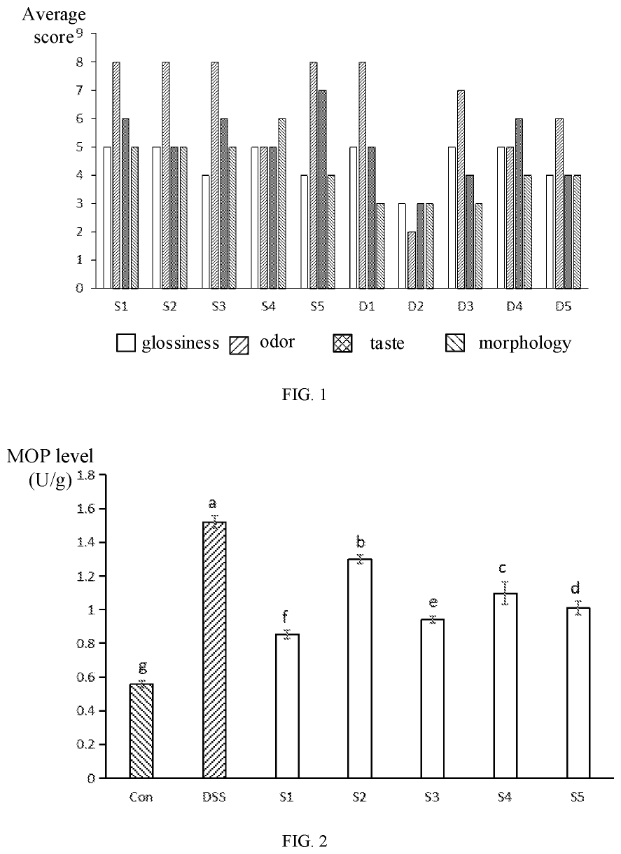

The sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

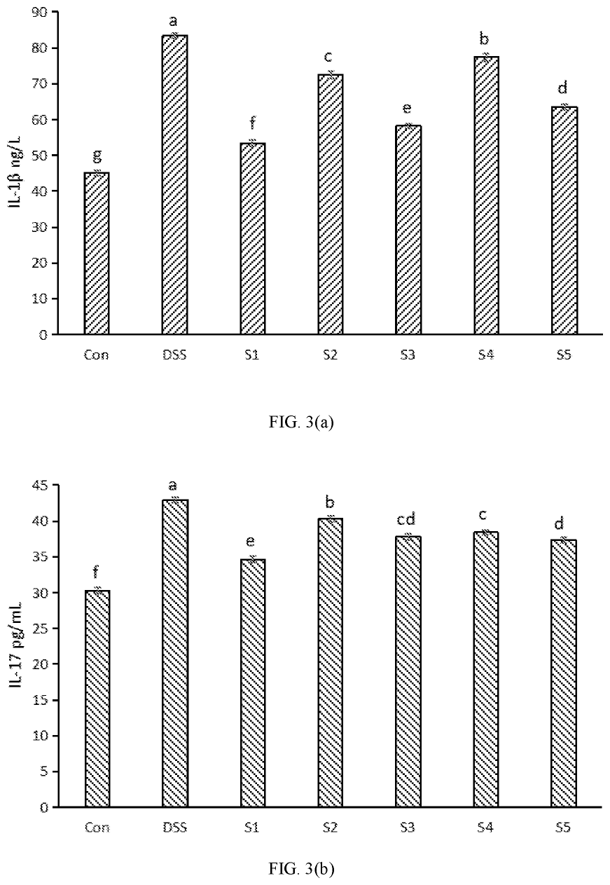

The inflammation-related genes IL-18 and IL-17 of RAW264.7 cells treated with the combination containing enriched HSO were detected by ELISA. The results are shown in FIG. 3 (a) and FIG. 3 (b).

### Embodiment 2 (S2)

The combination containing enriched HSO for preventing or alleviating IBD includes the following compositions, by weight percent:

40% of BCFA glycerides and 60% of HSO. The BCFA glycerides include: DAG and TAG, in a mass ratio of 1:1. Additionally, the mass ratio of anteiso-BCFAs to iso-BCFAs is 2:1.

The method of preparing the composition combination in the present embodiment is the same as that in Embodiment 1.

The fatty acid composition of a combination containing enriched HSO was detected and analyzed. The detection results are shown in Table 2.

The volatile flavor compositions of the combination containing enriched HSO are detected and analyzed, and the detection results are shown in Table 3.

Sensory evaluation and analysis were performed on a combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-Iβ and IL-17 were detected in RAW264.7 cells treated with a combination containing enriched HSO using ELISA. The results of the detection are shown in FIG. 3 (a) and FIG. 3 (b).

### Embodiment 3 (S3)

The combination containing enriched HSO for preventing or alleviating IBD includes the following compositions, by weight percent:

40% of BCFA glycerides and 60% of HSO are used in the mixture. The BCFA glycerides include: DAG and TAG, in a mass ratio of 1:1. Additionally, the mass ratio of anteiso-BCFAs to iso-BCFAs is 4:1.

The method of preparing the composition combination in the present embodiment is the same as that in Embodiment 1.

The fatty acid composition of the combination containing enriched HSO was detected and analyzed. The detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 of RAW264.7 cells treated with the combination containing enriched HSO were detected by ELISA. The results are shown in FIG. 3 (a) and FIG. 3 (b).

### Embodiment 4 (S4)

The combination containing enriched HSO for preventing or alleviating IBD includes the following compositions, by weight percent:

50% of BCFA glycerides and 50% of HSO are used in the mixture. The BCFA glycerides include: DAG and TAG, in a mass ratio of 2:1. Additionally, the mass ratio of anteiso-BCFAs to iso-BCFAs is 2:1.

The method of preparing the composition combination in the present embodiment is the same as that in Embodiment 1.

The fatty acid compositions of the combination containing enriched HSO were detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-Iß and IL-17 were detected in RAW264.7 cells treated with the combination containing enriched HSO using ELISA. The results are shown in FIG. 3 (a) and FIG. 3 (b).

Embodiment 5 (S5)

The combination containing enriched HSO for preventing or alleviating IBD includes the following compositions, by weight percent:

30% of BCFA glycerides and 70% of HSO are used in the mixture. The BCFA glycerides include: DAG and TAG, in a mass ratio of 0:2. Additionally, the mass ratio of anteiso-BCFAs to iso-BCFAs is 4:1.

The method of the preparing the composition combination in the present embodiment is the same as that in the Embodiment 1.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 were detected in RAW264.7 cells treated with the combination containing enriched HSO using ELISA. The results are shown in FIG. 3 (a) and FIG. 3 (b).

Comparative embodiment 1 (D1)

The combination containing enriched HSO for preventing or alleviating the IBD includes the following compositions, by weight percent:

15% of BCFA and 85% of HSO are used in the mixture. The BCFA glycerides include: DAG and TAG, in a mass ratio of 1:1. Additionally, the mass ratio of anteiso-BCFAs to iso-BCFAs is 3:1.

The method of the preparing the composition combination in the present embodiment is the same as that in the Embodiment 1.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 were detected in RAW264.7 cells treated with the combination containing enriched HSO using ELISA. The results are shown in FIG. 3 (a) and FIG. 3 (b).

Comparative embodiment 2 (D2)

The combination containing enriched HSO for preventing or alleviating the IBD includes the following compositions, by weight percent:

75% of BCFA glycerides and 25% of HSO are used in the mixture. The BCFA glycerides include: DAG and TAG, in a mass ratio of 1:1. Additionally, the mass ratio of anteiso-BCFAs to iso-BCFAs is 3:1.

The method of the preparing the composition combination in the present embodiment is the same as that in the Embodiment 1.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 were detected in RAW264.7 cells treated with the combination containing enriched HSO using ELISA. The results are shown in FIG. 3 (a) and FIG. 3 (b).

Comparative embodiment 3 (D3)

The combination containing enriched HSO for preventing or alleviating the IBD includes the following compositions, by weight percent:

40% of BCFA glycerides and 60% of HSO are used in the mixture. The BCFA glycerides include: DAG and TAG, in a mass ratio of 4:1. Additionally, the mass ratio of anteiso-BCFAs to iso-BCFAs is 3:1.

The method of the preparing the composition combination in the present embodiment is the same as that in the Embodiment 1.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 were detected in RAW264.7 cells treated with the combination containing enriched HSO using ELISA. The results are shown in FIG. 3 (a) and FIG. 3 (b).

Comparative embodiment 4 (D4)

The combination containing enriched HSO for preventing or alleviating the IBD includes the following compositions, by weight percent:

40% of BCFA glycerides and 60% of HSO are used in the mixture. The BCFA glycerides include: DAG and TAG, in a mass ratio of 1:1. Additionally, the mass ratio of anteiso-BCFAs to iso-BCFAs is 5:1.

The method of the preparing the composition combination in the present embodiment is the same as that in the Embodiment 1.

The fatty acid composition of the combination containing enriched HSO was detected and analyzed, and the detection results are shown in Table 2.

The volatile flavor compositions of the combination containing enriched HSO were detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-Iβ and IL-17 were detected in RAW264.7 cells treated with the combination containing enriched HSO using ELISA. The results are shown in FIG. 3 (a) and FIG. 3 (b).

Comparative embodiment 5 (D5)

The combination containing enriched HSO for preventing or alleviating the IBD includes the following compositions, by weight percent:

60% of BCFA glycerides and 40% of HSO are used in the mixture. The BCFA glycerides include: DAG and TAG, in a mass ratio of 3:1. Additionally, the mass ratio of anteiso-BCFAs to iso-BCFAs is 1:3.

The method of the preparing the composition combination in the present embodiment is the same as that in the Embodiment 1.

The fatty acid compositions of the combination containing enriched HSO were detected and analyzed, and the detection results are shown in Table 2.

The volatile flavor composition of the combination containing enriched HSO was detected and analyzed, and the detection results are shown in Table 3.

Sensory evaluation and analysis were performed on the combination containing enriched HSO, and the results of the evaluation and analysis are shown in FIG. 1.

MPO activity was detected and analyzed in the colon tissues of mice fed with the combination containing enriched HSO. The results are shown in FIG. 2.

The inflammation-related genes IL-18 and IL-17 were detected in RAW264.7 cells treated with the combination containing enriched HSO using ELISA. The results are shown in FIG. 3 (a) and FIG. 3 (b).

A proportion of each composition combination made in Embodiments 1 to 5 and in

Comparative Embodiments 1 to 5 is shown in Table 4.

TABLE 2
The fatty acid composition of the combination containing enriched HSO

| Fatty acid (mass %) | | Comparative Embodiment | | | | | | | | | |
|---|---|---|---|---|---|---|---|---|---|---|---|
| Embodiment | | S1 | S2 | S3 | S4 | S5 | D1 | D2 | D3 | D4 | D5 |
| C8:0 | | 0.03 | 0.12 | 0.00 | 0.57 | 0.03 | 0.00 | 6.78 | 0.02 | 3.92 | 1.05 |
| C10:0 | | 0.00 | 0.15 | 0.89 | 0.38 | 0.00 | 1.42 | 3.92 | 0.00 | 7.16 | 2.49 |
| C12:0 | | 0.12 | 0.00 | 0.34 | 0.66 | 0.42 | 0.00 | 2.43 | 0.24 | 0.00 | 2.02 |
| C13:0 | | 0.00 | 0.43 | 0.00 | 0.21 | 0.82 | 0.97 | 5.22 | 0.00 | 0.00 | 0.00 |
| C14:0 | | 0.08 | 0.38 | 0.75 | 0.86 | 0.67 | 3.84 | 8.45 | 0.37 | 0.00 | 2.77 |
| C14:1 | | 0.00 | 0.00 | 0.00 | 0.00 | 0.00 | 0.00 | 0.00 | 0.00 | 0.00 | 0.00 |
| C15:0 | | 0.31 | 0.21 | 0.18 | 1.14 | 0.00 | 0.06 | 3.89 | 0.06 | 5.87 | 0.92 |
| C16:0 | HSO | 5.46 | 5.46 | 5.46 | 4.55 | 6.37 | 7.74 | 2.28 | 5.46 | 5.46 | 3.64 |
| | BCFA glycerides | 0.43 | 0.58 | 0.76 | 0.84 | 0.00 | 0.00 | 0.17 | 0.00 | 1.28 | 3.92 |
| C16:1 | | 0.52 | 0.39 | 1.09 | 0.82 | 0.86 | 0.23 | 4.73 | 0.00 | 0.00 | 0.94 |
| C17:0 | | 0.28 | 0.25 | 0.58 | 0.87 | 0.00 | 2.89 | 6.98 | 0.08 | 4.83 | 0.68 |
| C17:1 | | 0.00 | 0.00 | 0.00 | 0.00 | 0.00 | 0.00 | 0.00 | 0.00 | 0.00 | 0.00 |
| C18:0 | HSO | 1.65 | 1.65 | 1.65 | 1.38 | 1.93 | 2.34 | 0.69 | 1.65 | 1.65 | 1.10 |
| | BCFA glycerides | 0.19 | 0.61 | 1.21 | 0.43 | 0.11 | 1.98 | 4.18 | 0.39 | 0.78 | 1.45 |
| C18:1 | HSO | 48.00 | 48.00 | 48.00 | 40.00 | 56.00 | 68.00 | 20.00 | 48.00 | 48.00 | 32.00 |
| | BCFA glycerides | 38.03 | 36.38 | 34.19 | 42.35 | 27.07 | 3.45 | 26.64 | 38.83 | 16.15 | 39.42 |

| | | | | | | | | | | |
|---|---|---|---|---|---|---|---|---|---|---|
| C18:2 | 4.48 | 4.48 | 4.48 | 3.73 | 5.22 | 6.34 | 2.02 | 4.48 | 4.48 | 2.98 |
| C18:3 | 0.16 | 0.16 | 0.16 | 0.13 | 0.18 | 0.22 | 0.07 | 0.16 | 0.16 | 0.10 |
| C20:0 | 0.00 | 0.49 | 0.00 | 0.86 | 0.02 | 0.15 | 1.44 | 0.00 | 0.00 | 4.35 |
| C20:1 | 0.26 | 0.26 | 0.26 | 0.22 | 0.30 | 0.37 | 0.11 | 0.26 | 0.26 | 0.17 |
| Saturated fatty acid | 8.55 | 10.33 | 11.82 | 12.75 | 10.37 | 21.39 | 46.43 | 8.27 | 30.95 | 24.39 |
| (SFA) | | | | | | | | | | |
| Mono-unsaturated fatty | 86.81 | 85.03 | 83.54 | 83.39 | 84.23 | 72.05 | 51.48 | 87.09 | 64.41 | 72.53 |
| acids (MUFA) | | | | | | | | | | |
| Poly-unsaturated fatty | 4.64 | 4.64 | 4.64 | 3.86 | 5.40 | 6.56 | 2.09 | 4.64 | 4.64 | 3.08 |
| acids (PUFA) | | | | | | | | | | |

As shown in Table 2, the fatty acid compositions of the above embodiments are reasonable. The BCFA glycerides are formed by proportionally compounding the DAG with the TAG. The combination containing enriched HSO can be easily digested, absorbed, and metabolized, alleviating fat digestion and absorption disorders in IBD patients. Additionally, the composition combinations in the above embodiments have enriched MUFA, making the combination containing enriched HSO table. This allows for the regulation of physiological functions in humans and improves metabolism.

TABLE 3
GC/MS analysis was performed to determine the volatile flavor
compositions of the combination containing enriched HSO

| Compound | Relative proportions (%) of volatile flavor | | | | | | | | | |
|---|---|---|---|---|---|---|---|---|---|---|
| name and | compositions of the combination containing enriched HSO | | | | | | | | | |
| class | S1 | S2 | S3 | S4 | S5 | D1 | D2 | D3 | D4 | D5 |

Alkanes

| | | | | | | | | | | | |
|---|---|---|---|---|---|---|---|---|---|---|---|
| 1 | Tetracosane | 15.25 | 13.64 | 14.93 | 14.22 | 15.15 | 14.35 | 12.46 | 15.16 | 13.82 | 15.35 |

Aldehydes

| | | | | | | | | | | | |
|---|---|---|---|---|---|---|---|---|---|---|---|
| 2 | Benzaldehyde | 2.00 | 1.85 | 1.97 | 2.08 | 2.04 | 1.98 | 1.67 | 2.08 | 1.76 | 2.04 |
| 3 | Phenylacetaldehyde | 9.00 | 8.86 | 9.05 | 8.98 | 9.12 | 8.71 | 8.02 | 8.94 | 8.51 | 9.02 |
| 4 | (Z)-14-Methyl-8- | 0.77 | 0.72 | 0.77 | 0.78 | 0.77 | 0.76 | 0.68 | 0.78 | 0.72 | 0.77 |
| | hexadecenoic | | | | | | | | | | |
| | acid-1-acetal | | | | | | | | | | |
| 5 | 2-Phenylcrotonaldehyde | 0.13 | 0.11 | 0.13 | 0.12 | 0.12 | 0.13 | 0.07 | 0.15 | 0.14 | 0.14 |

Acids

| | | | | | | | | | | | |
|---|---|---|---|---|---|---|---|---|---|---|---|
| 6 | 4-Methylcaprylic | 0.24 | 1.21 | 0.00 | 1.39 | 0.18 | 0.00 | 3.64 | 0.12 | 2.21 | 0.00 |
| | acid | | | | | | | | | | |
| 7 | 4-Ethyl caprylic acid | 0.17 | 0.62 | 0.00 | 2.46 | 0.25 | 0.00 | 4.15 | 0.26 | 2.24 | 0.00 |
| 8 | 2-Methylene- | 4.19 | 4.08 | 3.96 | 3.76 | 4.14 | 4.18 | 3.64 | 4.22 | 3.78 | 4.23 |
| | Cyclopropanecarboxylic | | | | | | | | | | |
| | acid | | | | | | | | | | |
| 9 | Nonanoic acid | 0.12 | 0.12 | 0.11 | 0.12 | 0.12 | 0.11 | 0.06 | 0.12 | 0.11 | 0.11 |
| 10 | 6-octadecenoic acid | 7.73 | 7.61 | 7.42 | 7.71 | 7.75 | 7.76 | 6.92 | 7.69 | 7.65 | 7.75 |
| 11 | Stearate | 2.39 | 3.06 | 4.88 | 2.17 | 2.52 | 5.57 | 6.45 | 2.52 | 3.72 | 1.86 |

Alcohols

| | | | | | | | | | | | |
|---|---|---|---|---|---|---|---|---|---|---|---|
| 12 | Trans-2-undecylenic- | 3.12 | 3.08 | 3.12 | 3.10 | 3.12 | 3.06 | 2.88 | 3.10 | 2.98 | 3.18 |
| | 1-ol | | | | | | | | | | |

Ketones

| | | | | | | | | | | | |
|---|---|---|---|---|---|---|---|---|---|---|---|
| 13 | 2-hydroxy-3-methyl- | 0.33 | 0.35 | 0.29 | 0.33 | 0.34 | 0.28 | 0.23 | 0.32 | 0.28 | 0.34 |
| | 2-cyclopentene-1- | | | | | | | | | | |
| | ketone | | | | | | | | | | |
| 14 | β-elemene | 0.23 | 0.23 | 0.23 | 0.24 | 0.23 | 0.22 | 0.17 | 0.24 | 0.22 | 0.28 |
| 15 | (+)-γ-Curjunene | 0.11 | 0.11 | 0.11 | 0.10 | 0.10 | 0.11 | 0.07 | 0.12 | 0.12 | 0.11 |
| 16 | Eremophilene | 0.63 | 0.64 | 0.63 | 0.65 | 0.63 | 0.62 | 0.54 | 0.65 | 0.62 | 0.64 |

Esters

| | | | | | | | | | | | |
|---|---|---|---|---|---|---|---|---|---|---|---|
| 17 | Ascorbyl dipalmitate | 2.31 | 2.33 | 2.31 | 2.31 | 2.32 | 2.28 | 1.96 | 2.36 | 2.06 | 2.32 |

Phenols

| | | | | | | | | | | | |
|---|---|---|---|---|---|---|---|---|---|---|---|
| 18 | Maltol | 2.52 | 2.61 | 2.57 | 2.52 | 2.53 | 2.48 | 2.11 | 2.58 | 2.48 | 2.52 |

Heterocycles

| | | | | | | | | | | | |
|---|---|---|---|---|---|---|---|---|---|---|---|
| 19 | 2-Methylpyrazine | 6.07 | 6.11 | 6.02 | 6.07 | 6.12 | 6.09 | 5.74 | 6.04 | 5.91 | 6.07 |
| 20 | Furaldehyde | 11.39 | 11.42 | 11.04 | 11.45 | 11.51 | 11.08 | 10.49 | 11.27 | 11.13 | 11.48 |
| 21 | 5-Methyl furfural | 16.31 | 16.25 | 16.41 | 15.67 | 16.28 | 15.94 | 14.68 | 16.28 | 15.47 | 16.42 |
| 22 | 4,6-Sulfadimidine | 3.56 | 3.53 | 3.25 | 3.25 | 3.14 | 3.34 | 3.09 | 3.60 | 3.25 | 3.66 |
| 23 | 2-ethyl-6- | 7.44 | 7.27 | 7.28 | 6.68 | 7.52 | 7.38 | 7.01 | 7.48 | 7.26 | 7.64 |
| | methylpyrazine | | | | | | | | | | |
| 24 | 2-Acetyl pyrrole | 0.97 | 0.89 | 0.79 | 0.84 | 0.96 | 0.81 | 0.74 | 0.92 | 0.93 | 1.08 |
| 25 | 2,5-dimethyl-3- | 0.02 | 0.02 | 0.03 | 0.02 | 0.03 | 0.02 | 0.00 | 0.02 | 0.02 | 0.02 |
| | Ethylpyrazine | | | | | | | | | | |
| 26 | €-2-methyl-6- | 0.29 | 0.31 | 0.25 | 0.28 | 0.31 | 0.27 | 0.22 | 0.26 | 0.26 | 0.29 |
| | propenylpyrazine | | | | | | | | | | |
| 27 | 2,3-(2H)-3,5- | 2.46 | 2.65 | 2.14 | 2.36 | 2.48 | 2.24 | 2.05 | 2.43 | 2.12 | 2.45 |
| | Dihydroxy-6-methyl- | | | | | | | | | | |
| | 4(4H)-Furaneol | | | | | | | | | | |

TABLE 4
Compositions of the combination containing enriched HSO

| | BCFA glycerides | | | |
|---|---|---|---|---|
| | Total proportion | | | |
| | of the | | | |
| | BCFA glycerides | DAG:TAG | anteiso-BCFAs:iso-BCFAs | HSO |
| S1 | 40% | 1:1 | 3:1 | 60% |
| S2 | 40% | 1:1 | 2:1 | 60% |
| S3 | 40% | 1:1 | 4:1 | 60% |
| S4 | 50% | 2:1 | 2:1 | 50% |
| S5 | 30% | 0:2 | 4:1 | 70% |
| D1 | 15% | 1:1 | 3:1 | 85% |
| D2 | 75% | 1:1 | 3:1 | 25% |
| D3 | 40% | 4:1 | 3:1 | 60% |
| D4 | 40% | 1:1 | 5:1 | 60% |
| D5 | 60% | 3:1 | 1:3 | 40% |

As shown in FIG. 1, the sensory evaluation score for the composition combinations in Embodiments 1 to 5 is higher than the score for the composition combinations in Comparative Embodiments 1 to 5. The composition combination made in Embodiment 1 received the highest sensory evaluation score. The score of the composition combination made in Comparative Embodiment 2 is much lower than that of the other Embodiments and Comparative Embodiments. Combined with Table 3, it can be seen that the composition combination used in the Comparative Embodiment 2 has a larger amount of 4-Methylcaprylic acid and 4-Ethyl caprylic acid. Both 4-Methylcaprylic acid and 4-Ethyl caprylic acid have an unpleasant goat stinky odor. Additionally, the HSO in the composition combination used in the Comparative Embodiment 2 is much lower than that in the composition combinations used in the other Embodiments and Comparative Embodiments. Therefore, the HSO is insufficient to eliminate the unpleasant odor caused by the BCFAs.

The MPO is a peroxidase found in neutrophils and is an indicator of neutrophil infiltration and acute inflammation. As seen in FIG. 2, the MPO activities of the colon tissues in the normal control group did not change significantly. In contrast, the MPO activities of the colon tissues in the induction modeling groups were significantly increased compared to those in the normal control group. In Embodiments 1 to 5, the MPO activities of the colon tissues in the intervention group, which were fed with the composition combination from Embodiment 1, were significantly decreased. The MPO activities of the colon tissues in the intervention groups, which were fed with the composition combinations from the Embodiments 3 and 5, were decreased to a greater extent.

IL-1β and IL-17 are involved in inflammation and immune response reactions and are important cytokines that reflect the severity of inflammation. As shown in FIG. 3, the expression levels of these cytokines in the supernatants of colon tissue cells from the normal control group did not change significantly. However, compared to the normal control group, the expression levels of these cytokines in the supernatants of colon tissue cells from the induction modeling group were significantly increased. In Embodiments 1 to 5, the composition combination of Embodiment 1, the composition combination of Embodiment 3, and the composition combination of Embodiment 5 all showed significant anti-inflammatory effects. The expression levels of the cytokines in the supernatants of colon tissue cells treated with the composition combination of Embodiment 2 and the composition combination of Embodiment 4 were decreased to a lesser extent. In summary, the effect of the composition combination in Embodiment 1 was better than that of the composition combination in Embodiment 3 and that of the composition combination in Embodiment 5; and the effects of the composition combinations in Embodiments 1, 3, and 5 were better than that of the composition combinations in Embodiment 2 and 4.

It is understood that the above shows only embodiments of the present disclosure and is not intended to limit the scope of the present disclosure. Any equivalent structure or equivalent process transformation performed based on the specification of the present disclosure, whether directly or indirectly applied in other technical fields, shall be included in the scope of the present disclosure.

1. A combination containing enriched hill shaddock oil (HSO) for preventing or relieving inflammatory bowel disease (IBD), wherein,
in mass percentages, the combination contains 30% to 50% of branched chain fatty acid (BCFA) glycerides and 50% to 70% of HSO.

2. The combination containing enriched HSO as claimed in claim 1, wherein the BCFA glycerides consist of a diacylglycerol (DAG) and a triacylglycerol (TAG), and a mass ratio of the DAG to the TAG is in a range of 0:3 to 2:1.

3. The combination containing enriched HSO as claimed in claim 2, wherein the mass ratio of the DAG to the TAG is 1:1.

4. The combination containing enriched HSO as claimed in claim 1, wherein the BCFA glycerides are fatty acids with 8 to 20 carbon atoms.

5. The combination containing enriched HSO as claimed in claim 4, wherein the fatty acids comprise anteiso-BCFAs and iso-BCFAs; a mass ratio of the anteiso-BCFAs to the iso-BCFAs is in a range of 2:1 to 4:1.

6. The combination containing enriched HSO as claimed in claim 4, comprising: fatty acids in following mass percentages: 80% to 90% oleic acid, 2% to 9% linoleic acid, 0.05% to 1.5% linolenic acid, 3% to 8% palmitic acid, and 1% to 5% stearic acid.

7. The combination containing enriched HSO as claimed in claim 6, comprising: fatty acids in following mass percentages: 82% to 87% of oleic acids, 3% to 7% linoleic acid, 0.1% to 1% linolenic acid, 4% to 7% palmitic acid, and 1% to 3% stearic acid.

8. The combination containing enriched HSO as claimed in claim 1, wherein, in mass percentages, volatile flavor compositions of the HSO comprise: 5% to 8% of 2-methylpyrazine; 6% to 10% of 2-ethyl-6-methylpyrazine; 9% to 14% of furaldehyde; 14% to 18% of 5-methyl furfural; 2% to 5% of 4, 6-sulfadimidine; 0.1% to 2% of 2-acetyl pyrrole; 10% to 20% of alkanes; 10% to 20% of aldehydes; 10% to 18% of acids; 2% to 5% of alcohols; 0.5% to 3% of ketones; 1% to 5% of phenols; and 1% to 5% of esters.

9. A method of preparing the combination containing enriched HSO as claimed in claim 2, comprising:
mixing TAG and DAG according to the mass ratio to obtain a first mixture, heating the mixture, stirring the mixture, and obtaining the BCFA glycerides;
and stopping the heating, adding the HSO to the BCFA glycerides to obtain a second mixture, stirring and cooling the second mixture to a room temperature to obtain the combination containing enriched HSO.

10. A method of preventing or alleviating IBD, comprising: applying a combination containing enriched hill shaddock oil (HSO) as fat to a patient with the inflammatory bowel disease (IBD); or incorporating the combination containing enriched HSO into food provided for a patient with the IBD;
wherein the combination containing enriched HSO, in mass percentages, contains: 30% to 50% of branched chain fatty acid (BCFA) glycerides and 50% to 70% of HSO.

11. The method as claimed in claim 10, wherein the BCFA glycerides consist of a diacylglycerol (DAG) and a triacylglycerol (TAG), and a mass ratio of the DAG to the TAG is in a range of 0:3 to 2:1.

12. The method as claimed in claim 11, wherein the mass ratio of the DAG to the TAG is 1:1.

13. The method as claimed in claim 10, wherein the BCFA glycerides are fatty acids with 8 to 20 carbon atoms.

14. The method as claimed in claim 13, wherein the fatty acids comprise anteiso-BCFAs and iso-BCFAs; a mass ratio of the anteiso-BCFAs to the iso-BCFAs is in a range of 2:1 to 4:1.

15. The method as claimed in claim 13, wherein the combination containing enriched HSO comprises: fatty acids in following mass percentages: 80% to 90% oleic acid, 2% to 9% linoleic acid, 0.05% to 1.5% linolenic acid, 3% to 8% palmitic acid, and 1% to 5% stearic acid.

16. The method as claimed in claim 15, wherein the combination containing enriched HSO comprises fatty acids in following mass percentages: 82% to 87% of oleic acids, 3% to 7% linoleic acid, 0.1% to 1% linolenic acid, 4% to 7% palmitic acid, and 1% to 3% stearic acid.

17. The method as claimed in claim 10, wherein in mass percentages, volatile flavor compositions of the HSO comprise: 5% to 8% of 2-methylpyrazine; 6% to 10% of 2-ethyl-6-methylpyrazine; 9% to 14% of furaldehyde; 14% to 18% of 5-methyl furfural; 2% to 5% of 4, 6-sulfadimidine; 0.1% to 2% of 2-acetyl pyrrole; 10% to 20% of alkanes; 10% to 20% of aldehydes; 10% to 18% of acids; 2% to 5% of alcohols; 0.5% to 3% of ketones; 1% to 5% of phenols; and 1% to 5% of esters.
